(19) United States
(12) Patent Application Publication
(10) Pub. No.: US 2021/0386302 A1
(43) Pub. Date: Dec. 16, 2021

(54) Optical Sensor Probe and Method for Measuring Blood Flow Rate, Blood Viscosity and Vascular Elastic Modulus Using the Optical Sensor Probe

(71) Applicant: Nippon Telegraph and Telephone Corporation, Tokyo (JP)

(72) Inventor: Nobutatsu Koshoubu, Musashino-shi, Tokyo (JP)

(21) Appl. No.: 17/295,717

(22) PCT Filed: May 15, 2019
(86) PCT No.: PCT/JP2019/019404

§ 371 Date: May 20, 2021

(30) Foreign Application Priority Data
Dec. 20, 2018 (JP) 2018-238803

(51) Int. Cl. A61B 5/026; A61B 5/02; A61B 5/00

(52) U.S. Cl. CPC A61B 5/0261; A61B 5/02035; A61B 2562/0242; A61B 5/6843; A61B 2562/0238; A61B 5/02007

(57) ABSTRACT

An optical sensor probe in which one end of optical fibers, of which the other end is connected to a light source or a Doppler measurement device, is linearly supported and arranged in a section of a buckling length L, a movement of the optical fibers in an optical axis direction is restricted at a fiber fixing point on an optical fiber-proximal side in the section of the buckling length L, the optical fibers are protrusively arranged through a restriction hole on an optical fiber-distal side in the section of the buckling length L, and the optical fibers are supported so as to be allowed to move only in the optical axis direction through the restriction hole. A measurement method is capable of measuring a blood flow rate, blood viscosity, or a vascular elastic modulus of the measurement target by measuring a Doppler shift of scattered light from the measurement target due to light emitted from the light source.

### TECHNICAL FIELD

The present invention relates to the technical fields of an optical sensor probe and measurement methods for a blood flow rate and blood viscosity in the subcutis of an animal, a human body, or the like and for an elastic modulus (Young's modulus) of capillaries in the skin using the optical sensor probe. Qualitative changes in blood flow rate, blood viscosity, and vascular elastic modulus can be simultaneously evaluated by measuring the change in blood flow rate at the time of pressure change. This can be particularly used as a method for evaluating the state of blood viscosity or a vascular elastic modulus used in preventive medicine, initial treatment, or the like.

### BACKGROUND ART

Description of Current Technology

Importance of Measurement of Blood Flow Rate

As an optical sensor probe for measuring a flow rate of a fluid and a flow rate measurement method in which the optical sensor probe is used, an optical sensor probe which is a measurement system capable of directly measuring a blood flow rate without performing any processing such as shaving of the hair for the skin where a lot of hair grows and a flow rate measurement method in which such an optical sensor probe is used are required when measuring a blood flow rate in the subcutis of an animal, a human body, or the like, for example. Such an optical sensor probe and flow rate measurement method can be used for measuring blood flows in various kinds of subcutis used in the veterinary medicine field or the medical field for a human body, particularly in the beauty field as a measure against hair loss on the human scalp.

Examples of Diseases in which Elasticity of Blood Vessels Changes

In Japan, the onset risk of lifestyle-related diseases is currently increasing due to the influence of the Westernization of dietary habits, aging, stress, or the like. This risk is considered to have risk factors such as (1) hypertension, (2) abnormal blood lipids including cholesterol, (3) diabetes, (4) aging (male: 45 years of age or older, female: postmenopausal), (5) smoking, (6) obesity, (7) lack of exercise, (8) emotionally stressed state, (9) unbalanced diet, and (10) items of personal preference (such as alcohol, coffee, and tea). It is known that these risk factors promote arteriosclerosis and development of serious life-threatening diseases such as cerebrovascular disorders or heart disease. In the early state of arteriosclerosis, the blood vessel wall loses its elasticity and becomes hard. Plaque gradually develops in blood vessels, and the inside of the blood vessels becomes narrower. Eventually, the plaque becomes thrombi which obstruct blood flow. Blood flow supply to important organs is reduced in these processes, resulting in various diseases due to circulatory disorders.

From the above-described viewpoints, there is a growing need for inspections for measuring the elasticity of blood vessels using an optical sensor as a simple, non-invasive method.

### Importance of Measurement of Vascular Elastic Modulus

Furthermore, it is extremely important to promptly find an increase in vascular elastic modulus (Young's modulus) in the early stage of arteriosclerosis from the viewpoint of preventive medical care or initial medical care. In addition, it is important that the method is a simple, non-invasive method that can be carried out even at home.

If change in vascular elastic modulus (Young's modulus) can be evaluated through a simple, non-invasive method, an indicator of the condition of blood vessels can be obtained at home without having a check-up at a medical institution, which is extremely beneficial.

### Main Influences on Blood Viscosity

On the other hand, the blood flowing inside the blood vessels is composed of cellular components and liquid components. The cellular components of the blood account for about 45% of the total, and are composed of: red blood cells accounting for most of the cellular components; white blood cells; platelets; and the like. In addition, plasma is a liquid component of blood, 91% of the plasma is water, and solid components accounting for 9% of the plasma are dissolved in the water.

Examples of the main influences on the viscosity of blood include the protein concentration in plasma, the amount of red blood cells (hematocrit value: the ratio of the volume of red blood cells to that of whole blood), and the deformability of red blood cells. For this reason, the blood viscosity increases due to (1) an increase in the number of red blood cells, (2) an increase in concentration of crystalline proteins, and (3) a decrease in the amount of water in blood.

### Importance of Blood Viscosity

It is known that, in a case where the blood viscosity increases, that is, the fluidity decreases, thrombi are easily formed, whereby the risk of myocardial infarction or cerebral infarction increases. Therefore, the blood viscosity is one of important parameters having an influence on arteriosclerosis.

Examples of Diseases in which Blood Viscosity Increases

In addition, examples of diseases in which blood viscosity increases include hyperlipidemia, diabetes, blood hyperviscosity syndrome, polycythemia, and the like.

For this reason, it is possible to obtain an indicator of the progress of these diseases by evaluating the blood viscosity in an examination and in follow-up observations for the diseases exemplified above.

### Importance of Evaluation of Blood Viscosity and Vascular Elastic Modulus

From the above-described viewpoints, there is a growing need for measuring blood viscosity and a vascular elastic modulus (Young's modulus) in addition to a blood flow rate through a simple, non-invasive method.

### CITATION LIST

### Non Patent Literature

[NPL 1] Kenkichi Ohba et al., “Local Velocity Measurement of Opaque Fluid Flow Using Laser Doppler Velocimeter With Optical Dual Fiber Pickup”, Transactions of the Japan Society of Mechanical Engineers (Series B), Vol. 49, No. 447 (1983-11), pp. 2380-2389
[NPL 2] Masaru Kobayashi, et al., “Injection Molded Plastic Multifiber Connector Realizing Physical Contact with Fiber Elasticity”, IEEE JOURNAL OF SELECTED TOPICS IN QUANTUM ELECTRONICS, Vol. 5, No. 5, SEPTEMBER/OCTOBER 1999, pp. 1271-1277

### SUMMARY OF THE INVENTION

### Technical Problem

The present invention has been made in consideration of such circumstances, and an object of the present invention is to measure a blood flow rate of blood vessels in the epidermis or the like, a vascular elastic modulus, or a viscosity of blood flowing through blood vessels, using a relatively simple method.

At this time, it is an object of the present invention to develop a measurement method which is in an easy-to-use form and in which the measurement is as non-invasive as possible without imposing any burden on a subject of the measurement.

### Means for Solving the Problem

An example of an embodiment of the present invention includes the following configurations to achieve such an object.

Configuration 1

An optical sensor probe including: optical fibers of which one end is connected to a light source or a Doppler measurement device measuring a flow velocity using a laser Doppler, in which, at the other end, the optical fibers are linearly supported and arranged in a section of a buckling length L in a case where an optical sensor probe is in a non-measurement state, fixed to a fiber fixing point on an optical fiber-proximal side in the section of the buckling length L so that a movement of the optical fibers is restricted thereat, and arranged so that a distal end of the optical fibers protrudes through a restriction hole on an optical fiber-distal side in the section of the buckling length L, and, at the other end, in a case where the optical sensor probe is in a measurement state, the distal end of the protruding optical fibers abuts on a measurement target, the optical fibers are allowed to move only in a linear direction connecting the fiber fixing point to a center of the restriction hole by a protrusion length ΔL when pushed through the restriction hole, and the optical fibers in the section of the buckling length L buckle and maintain a pressing force between the measurement target and the distal end of the optical fibers by an elastic force.

Configuration 2

The optical sensor probe according to Configuration 1, in which, when a fiber diameter of the optical fibers is d, the protrusion length ΔL is smaller than a threshold value ΔLc given by an equation below, and the pressing force between the distal end of the optical fibers of the optical sensor probe and the measurement target can be adjusted with high accuracy by adjusting the protrusion length ΔL of the optical fibers.

ΔL_{c}=½(\sqrt{L²+π²d²}−L)

Configuration 3

The optical sensor probe according to Configuration 1, in which, when the fiber diameter of the optical fibers is d, the protrusion length ΔL is larger than a threshold value ΔLc given by an equation below, and the pressing force between the distal end of the optical fibers of the optical sensor probe and the measurement target can be adjusted with high accuracy by adjusting the buckling length L of the optical fibers.

ΔL_{c}=½(\sqrt{L²+π²d²}−L)

Configuration 4

The optical sensor probe according to any one of Configurations 1 to 3, in which a plurality of optical fibers or multi-core fibers are used as the optical fibers.

Configuration 5

The optical sensor probe according to Configuration 4, in which the light source or the Doppler measurement device is provided with a light switching function or a multiple simultaneous measurement function so that at least one fiber or core for light emission or light reception can be switched, or two or more optical fibers can perform simultaneous light emission or light reception.

Configuration 6

The optical sensor probe according to Configuration 4, in which at least one optical fiber at the distal portion of the optical sensor probe is exposed, and a member having at least one hole for inserting the exposed optical fibers can be attached to the optical sensor probe as an adapter socket.

Configuration 7

The optical sensor probe according to any one of Configurations 1 to 6, in which a plurality of light wavelengths are simultaneously or selectively used.

Configuration 8

A measurement method for a blood flow rate, the method including: measuring a Doppler shift of scattered light from the measurement target due to light emitted from the light source, using the Doppler measurement device to measure the blood flow rate of the measurement target using the optical sensor probe according to any one of Configurations 1 to 7.

Configuration 9

A measurement method for blood viscosity, the method including: measuring the blood viscosity from the blood flow rate and a pulse wave amplitude which change due to the adjustment of the pressing force of the optical sensor probe in the measurement method for a blood flow rate according to Configuration 8.

Configuration 10

The measurement method for blood viscosity according to Configuration 9, the method including: controlling a buckling length of the optical fibers and changing the pressing force of the optical sensor probe at the distal end of the optical fibers to measure the blood viscosity from a ratio of the blood flow rate to the pulse wave amplitude which changes.

Configuration 11

A measurement method for a vascular elastic modulus, the method including: adjusting and changing the pressing force of the optical sensor probe to measure a vascular elastic modulus from the obtained blood flow rate and pulse wave amplitude in the measurement method for a blood flow rate according to Configuration 8.

Configuration 12

The measurement method for a vascular elastic modulus according to Configuration 11, in which the vascular elastic modulus is measured from a proportional coefficient of a relationship between a ratio of the blood flow rate to a square root of the pulse wave amplitude and the pressing force of the optical sensor probe.

### Effects of the Invention

As described above, according to the present invention, it is possible to easily measure a blood flow rate, blood viscosity, and a vascular elastic modulus with accuracy.

### BRIEF DESCRIPTION OF DRAWINGS

FIG. 1 is a diagram illustrating a measurement principle of a laser Doppler flowmeter in the related art.

FIG. 2 is a diagram illustrating time dependence of an optical beat signal measured with the laser Doppler flowmeter in the related art.

FIG. 3 is a diagram illustrating frequency dependence (power spectrum: P(f)) of an optical beat signal.

FIG. 4 is a diagram illustrating frequency dependence of f·P(f).

FIG. 5 is a principle diagram of a pressing force generation structure utilizing fiber buckling of an optical sensor probe of an embodiment of the present invention.

FIG. 6 is a schematic diagram of the optical sensor probe of the embodiment of the present invention.

FIG. 7 is a diagram showing dependence of a pressing force on a fiber buckling length of the optical sensor probe of the embodiment of the present invention.

FIG. 8 is a schematic diagram of a distribution of scattered return light and laser light of the optical sensor probe of the embodiment of the present invention in which two fibers are used.

FIG. 9 is a schematic diagram showing an example of a specific aspect of the optical sensor probe of the embodiment of the present invention.

FIG. 10 is a diagram showing results obtained by measuring dependence of a blood flow rate on a probe pressure at a position of the middle finger pad of the hand using the optical sensor probe of the embodiment of the present invention.

FIG. 11 is a diagram showing results obtained by measuring dependence of a blood flow rate on a probe pressure at positions on the left and right forehead using the optical sensor probe of the embodiment of the present invention.

FIG. 12 is a diagram showing results obtained by measuring dependence of a blood flow rate on a probe pressure at the top of the head using the optical sensor probe of the embodiment of the present invention.

FIG. 13 shows cross-sectional views of the optical sensor probes of the embodiment of the present invention having different core center intervals between optical fibers.

FIG. 14 is a diagram showing results obtained by measuring dependence of a measurement value of a blood flow rate on a fiber interval at a probe distal end using the optical sensor probe of the embodiment of the present invention.

FIG. 15 is a diagram showing a time-varying waveform of a blood flow rate of the middle finger pad measured using the optical sensor probe of the embodiment of the present invention.

FIG. 16 is a schematic diagram illustrating a state of a subcutaneous blood vessel modified due to a pressing force.

FIG. 17 is a diagram showing dependence of a blood flow rate on a pulse wave amplitude measured at a position of the middle finger pad of the hand using the optical sensor probe of the embodiment of the present invention.

FIG. 18 is a diagram showing dependence of a blood flow rate on a pulse wave amplitude measured at positions on the left and right forehead using the optical sensor probe of the embodiment of the present invention.

FIG. 19 is a diagram showing dependence of a blood flow rate on a pulse wave amplitude measured at the top of the head using the optical sensor probe of the embodiment of the present invention.

FIG. 20 is a diagram showing dependence of a ratio of a blood flow rate to a square root of a pulse wave measured at a position of the middle finger pad of the hand and at the top of the head on a pressing force using the optical sensor probe of the embodiment of the present invention.

### DESCRIPTION OF EMBODIMENTS

First, the measurement principle of a laser Doppler flowmeter in the related art will be described as a premise of the present invention (refer to NPL 1).

Laser Doppler Flowmeter

The blood flow rate is evaluated through a laser Doppler method as a measurement method for a blood flow rate using an optical sensor probe of the embodiment of the present invention.

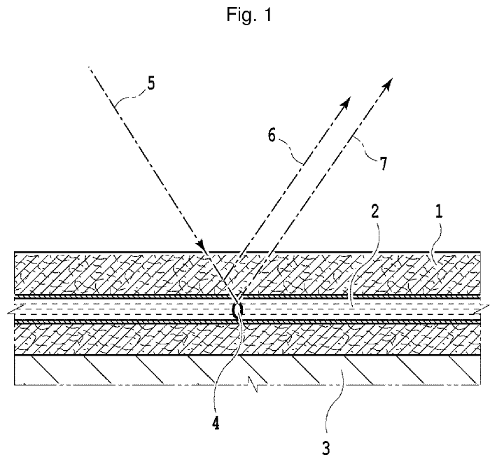

The measurement principle of the laser Doppler flowmeter in the related art will be described below with reference to FIG. 1 using an example of a case of measuring a flow velocity of blood flowing through capillaries under the skin of a human body, that is, a blood flow rate. In FIG. 1, it is assumed that the skin of a human body is, for example, a relatively thin scalp 1 in which a capillary 2 is present. The underneath of the capillary is supported by the periosteum and a skull 3.

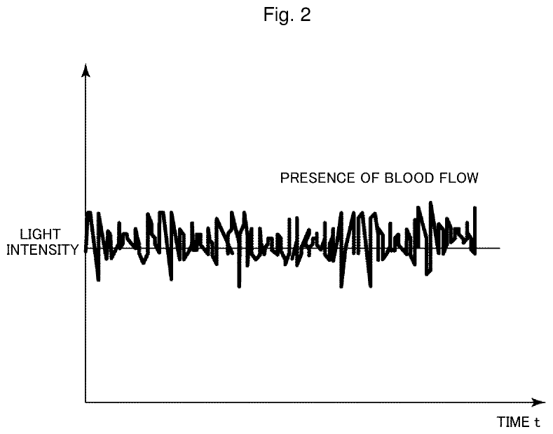

In a case where laser light 5 having a constant frequency f₀ is incident on the stationary scalp 1, the laser light incident on the inside of the skin of the scalp 1 causes (multiple) scattering. At this time, the components of scattered (return) light include scattered light 6 from stationary tissue in the scalp 1 and scattered light 7 from a red blood cell 4 moving in the capillary 2. The frequency of the scattered light 6 from the stationary tissue is f₀ which is the same as that of incident light 5. However, the scattered light 7 from the red blood cell 4 receives a Doppler shift due to the moving speed of the red blood cell 4, that is, the blood flow velocity, and the frequency is shifted to f₀+Δf. For this reason, the entire scattered (return) light becomes an optical beat signal in which the components of frequencies fc and f₀+Δf interfere with each other and the light intensity fluctuates over measurement time t as in FIG. 2.

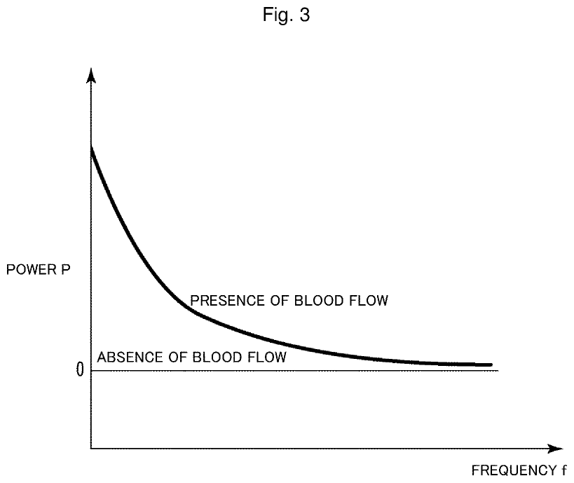

By Fourier-transforming the time-varying optical beat signal of FIG. 2, a power spectrum P(f) of the light intensity with respect to the frequency f is obtained as shown in FIG. 3. P(f) is constant at zero (a base line of “no blood flow”) in the absence of a blood flow, but indicates dependence on the frequency f in the presence of a blood flow. In general, P(f) becomes a curve in which the light intensity is large on a low frequency side and decreases along with an increase in frequency as shown in “presence of blood flow” of FIG. 3.

At this time, the value of the power spectrum P(f) of the intensity with respect to each frequency f is proportional to the amount of blood components having a velocity corresponding to the flow velocity of red blood cells causing a Doppler shift f.

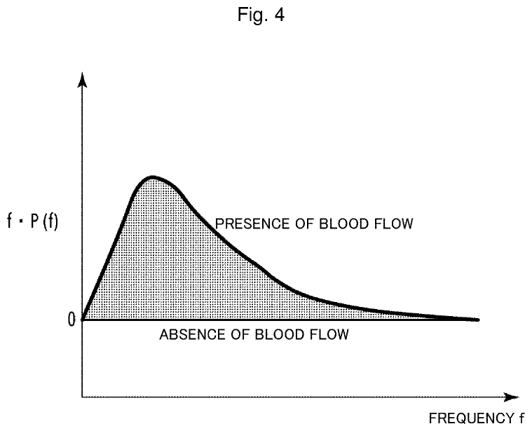

Next, in a case where f·P(f) is weighted with respect to the frequencies, a curve passing through the origin as shown in FIG. 4 is obtained. That is, a primary moment of P(f) with respect to the frequency f is calculated. Here, because the product of the flow velocity and the amount of components is a flow rate, the area (Equation (1) below) surrounded by the curve of f·P(f) and the straight line of f·P(f)=0, that is, the sum of flow velocity of blood×amount of components is proportional to the blood flow rate.

∫f·P(f)df (1)

Actually, when <I²> is the total power of a received light signal, an integral value is standardized by the total power <I²> of the received light signal so as not to depend on the difference in light receiving intensity. Therefore, the blood flow rate is represented by Expression (2) below.

$$Blood flow rate ∝ \frac{\int f \cdot P ( f ) df}{< {I}^{2} >} \qquad ( 2 )$$

In addition, as the measurement method for a blood flow rate, there is, for example, an ultrasonic Doppler method in which measurement of a flow rate is performed on the same principle using an ultrasonic wave with a constant frequency or a radar Doppler method using single-frequency electromagnetic waves, in addition to the measurement method for a flow rate performed through the laser Doppler method using laser light.

In addition to measuring only a value at one measurement point, a two-dimensional blood flow meter which detects optical beat signals at a plurality of measurement points with a two-dimensional CCD or a photodiode (PD) of a CMOS array sensor and detects blood flow rates by superimposing the blood flow rates at a plurality of measurement points on the captured image has also been developed.

Pressure Adjustment through Fiber Buckling

However, in order to measure the blood flow rate in the scalp, it is necessary to evaluate the blood flow rate in an environment in which a pressing force is adjusted as described above so as to prevent a blood-blocked state due to crushed subcutaneous tissue due to the influence of the pressing force of the optical probe on the skin while avoiding hair. For this reason, in the embodiment of the present invention, the blood flow rate of the scalp can be simply measured at a large signal-to-noise ratio by generating an appropriate pressing force through buckling of optical fibers themselves of the optical probe.

Hereinafter, a method for generating and adjusting a pressing force through fiber buckling in the embodiment of the present invention will be described.

Principle of Pressing Force Generation Structure

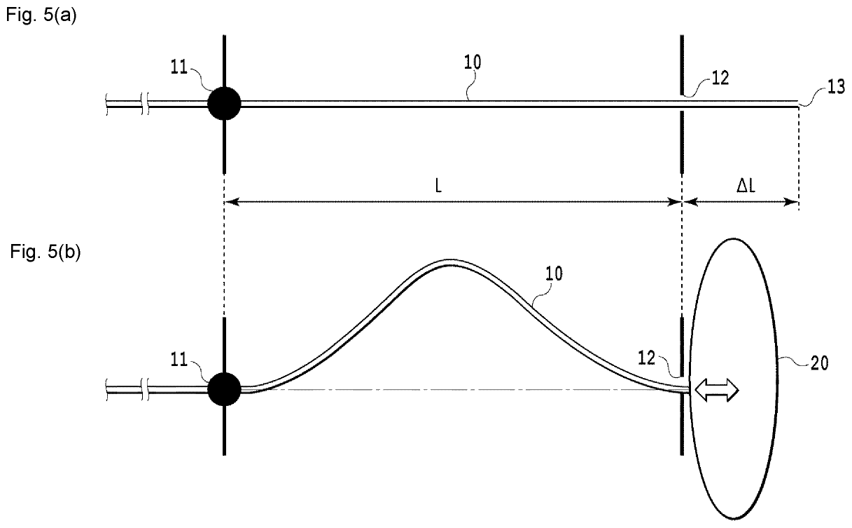

FIG. 5 shows a principle diagram of a pressing force generation structure of a pressing optical probe (optical sensor probe) of the embodiment of the present invention at a distal end of fibers in which a buckling force of the optical fibers is used.

FIG. 5(a) is a diagram showing a non-measurement state of the optical sensor probe. In the non-measurement state of the optical sensor probe, optical fibers 10 of which one end is connected to a light source or a Doppler measurement device which is at a left end and not shown in the drawing are linearly supported and arranged on the left and right of a section of a buckling length L on the other end side. The optical fibers 10 are fixed to a fiber fixing point 11 on an optical fiber-proximal side at a left end of the section of the buckling length L so that a movement of the optical fibers in an optical axis direction is restricted thereat.

In addition, the optical fibers 10 are arranged so that a distal end 13 of the optical fibers 10 protrudes by a protrusion length ΔL through a pinhole (restriction hole) 12 on an optical fiber-distal side on the right side of the section of the buckling length L. The optical fibers 10 are supported and arranged so that the movement (passing) of the fibers is allowed only in the optical axis direction (the direction of the fiber fixing point 11) through the pinhole (restriction hole) 12.

FIG. 5(b) is a diagram showing a measurement state of the optical sensor probe. In the measurement state of the optical sensor probe, the distal end 13 of the protruding optical fibers 10 abuts on a measurement target 20 such as the scalp to generate a pressing force. Then, the protruding portion of the optical fibers 10 with the length ΔL on the distal side moves to the proximal side through the pinhole (restriction hole) 12, and the section of the buckling length L of the optical fibers 10 buckles as shown in FIG. 5(b) and maintains a pressing force with the measurement target 20 using an elastic force. The measurement target 20 pushes the protruding portion (protrusion length ΔL) of the optical fibers 10 into the pinhole (restriction hole) 12 until the protruding portion thereof abuts on a member constituting the pinhole (restriction hole) 12 as much as possible, and stops in a state where a predetermined pressing force F is generated between the distal end 13 of the optical fibers 10 and the measurement target.

Laser light 5 for measurement supplied from a light source not shown in the drawing is emitted from the protruding distal end 13 of the optical fibers 10 to the measurement target 20. Conversely, scattered light 6 and 7 from the measurement target 20 is received at the distal end 13 of the optical fibers, incident on the optical fibers 10, and input to a laser Doppler measurement device not shown in the drawing. In the laser Doppler measurement device, the blood flow rate is measured through measuring a Doppler shift of received scattered light according to the principle of the laser Doppler flowmeter.

Magnitude of Pressing Force due to Fiber Buckling

In the pressing force generation structure of FIG. 5, buckling of the fibers 10 is possible only between the fiber fixing point 11 and the pinhole (restriction hole) 12, and the tip of the pinhole 12 has a fiber buckling structure so as to allow only protruding of the optical fibers 10 on a straight line.

Since the pinhole (restriction hole) 12 has a structure in which the optical fibers 10 can be freely put in and out (moved), the pressing force F generated per optical fiber when the optical fibers 10 are pushed by the measurement target 20 abutting on the distal end 13 of the optical fiber probe by the protrusion length ΔL can be theoretically calculated as a mechanical stress of an elastic body using the optical fibers as a cylindrical rod.

At this time, when the Young's modulus of the optical fibers is E, a cross section secondary moment is I, the fiber diameter is d, the buckling length is L, the protrusion length is ΔL, and the pressing force is F, the pressing force F represented by Equation (4) below is generated in a case of (A) ΔL<ΔLc on the basis of the threshold value ΔLc represented by Equation (3) below.

$$\begin{matrix}Δ {L}_{c} = \frac{1}{2} ( \sqrt{{L}^{2} + {π}^{2} {d}^{2}} - L ) & ( 3 ) \\ F = \frac{π E {d}^{2}}{4} \frac{Δ L}{L + Δ L} & ( 4 )\end{matrix}$$

Since ΔL<<L is usually satisfied, it can be said that the pressing force F is approximately proportional to the protrusion length ΔL within the range of ΔL<ΔLc. In addition, it is known that the pressing force F represented by Equation (5) below is generated in a case of (B) ΔL>ΔLc.

$$F = \frac{4 {π}^{2} EI}{{( L + Δ L )}^{2}} \qquad ( 5 )$$

That is, in the case where the protrusion length ΔL of the pushed fibers is larger than ΔLc of Equation (3), the pressing force F is approximately proportional to the −2 power of the buckling length L not to the protrusion length ΔL. In the embodiment of the present invention, the point that the pressing force F of the fibers generated under the condition of ΔL>ΔLc is approximately defined by the buckling length L as shown in Equation (5) is used for adjusting the pressing force.

Specific Structure of Optical Sensor Probe

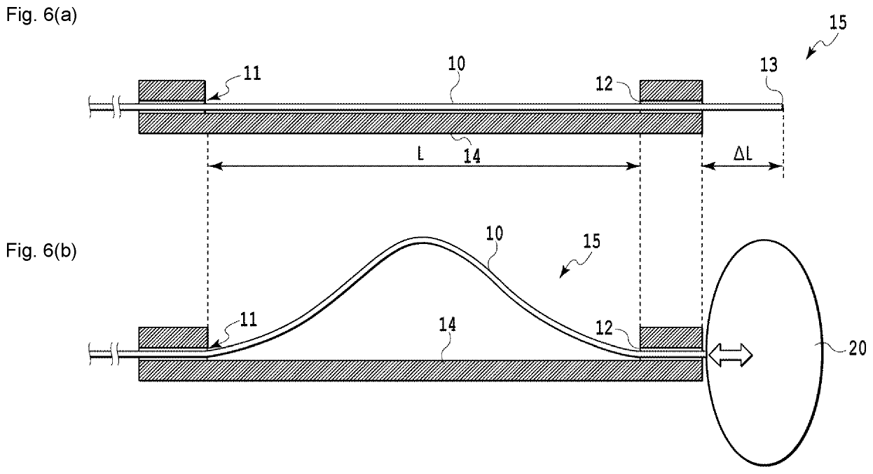

FIG. 6 is a schematic diagram which shows a specific structure of a pressing optical probe (optical sensor probe) 15 that generating a pressing force through fiber buckling and is the embodiment of the optical sensor probe of the present invention. Similar to FIG. 5, FIG. 6(a) is a view showing a non-measurement state of the optical sensor probe 15 and FIG. 6(b) is a view showing a measurement state of the optical sensor probe 15.

In FIG. 6(a), the optical fibers 10 having a buckling length L are linearly supported by the space between the fiber fixing point 11 and the pinhole (restriction hole) 12 which are provided at both ends of a fiber support portion 14 formed of, for example, a metal rod.

The fiber fixing point 11 and the pinhole (restriction hole) 12 are formed such that fibers 10 are passed through two short pipe-shaped members having side surfaces attached to both ends of the metal rod of the fiber support portion 14, for example. The optical fibers 10 on the right side of the pinhole (restriction hole) 12 are cut leaving a protrusion length ΔL to form the distal end 13 of the optical probe, and the optical fibers 10 in the pipe of the fiber fixing point 11 are fixed to the fiber support portion 14.

An optical sensor probe in which a pressing force is generated due to fiber buckling in the same principle as that in FIG. 5 is produced by pressing the distal end 13 of the optical probe against a measurement sample 20 as shown in FIG. 6(b). By pressing the distal end 13 of the optical fibers 10 by the protrusion length ΔL, the optical fibers 10 buckle over the buckling length L, and the pressing force F shown in Equation (5) is generated at the distal end of the optical fibers.

At this time, the pressing force F is obtained by utilizing a repulsive force generated by an elastic force of the optical fibers themselves and buckling in the section of the buckling length L in a case where the optical fibers are considered to be a cylindrical elastic body having a Young's modulus E, and the same structure as that of FIG. 1 can be produced as long as a rod-shaped elastic body is used instead of the optical fibers. For this reason, it is also possible to, for example, use optical fibers for only the section of the protrusion length ΔL to separate the section of the protrusion length from the buckling section where the pressing force F is generated, or attach each device for light emission or light reception such as laser diode (LD) or a photodiode (PD) to the distal end of the section of the protrusion length ΔL.

In addition, single-mode fibers (SMF), multi-mode fibers (MMF), step index fibers, graded index fibers (GI fibers), dispersion compensation fibers, fused quartz fibers, polymer-coated fibers, hole-assisted fibers (HAF), photonic crystal fibers (PCF), tape fibers, and polarization-maintaining fibers (PMF, PANDA fibers) can be used as the optical fibers as long as the optical fibers can be made to buckle as an elastic body having a Young's modulus E, and GI fibers, hole-assisted fibers (HAF), and photonic crystal fibers (PCF) are desirable from the viewpoint that propagation loss due to fiber bending such as buckling hardly fluctuates.

In addition, as the optical fibers to be used, two optical fibers may be used corresponding to light emission and light reception. However, the blood flow rate can be measured using only one optical fiber by emitting one polarized light component and receiving scattered light from a measurement object as another polarized light component using a polarization-maintaining fiber, and separating the polarized light components from each other using a polarization separation element.

Measurement of Pressing Force due to Fiber Buckling

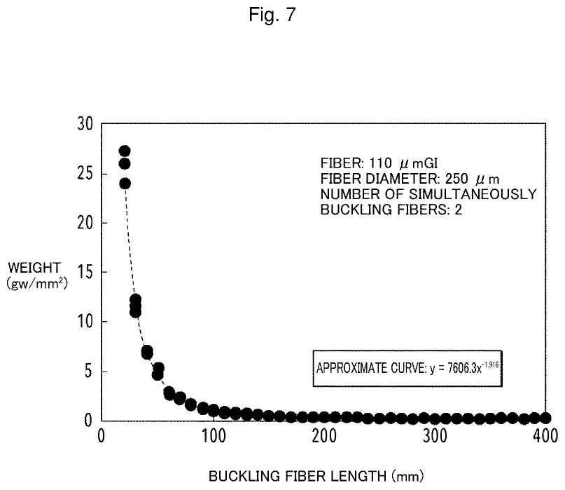

Results obtained by measuring dependence of the pressing force of the distal end of the optical sensor probe 15 on the buckling fiber length L are shown in FIG. 7. Here, the pressing force is expressed as a weight in a unit of pressure (gw/mm²), and protrusion length ΔL>ΔLc is satisfied. A bundle of two 250 micron diameter fibers having a 110 micron diameter graded index core is used as fibers for buckling on the assumption of actual measurement of a blood flow rate. As a result, the magnitude of the pressing force at the pressing force distal end which is generated through fiber buckling corresponds to two fibers, but decreases as the buckling length L of the fibers becomes longer as shown in Equation (5).

In a case where an approximate curve of the measurement results of FIG. 7 is calculated, the result is F=7606.3×L^{-1.916}. It can be seen that the result is extremely well matched with the −2 power of L in the theoretical equation shown in Equation (5).

It can be seen from the results of FIG. 7 that a weak pressing force can be adjusted with high accuracy by making the fiber fixing point 11 movable along the fiber support portion 14 and making the fiber buckling length L variable. For example, in a case where it is desired to adjust to a load of 2 gw or less, the length of the fibers to be made to buckle under the conditions of FIG. 7 may be 50 mm or longer.

Description of Implementation Configuration of Optical Sensor Probe

Hereinafter, implementation configurations of each of optical sensor probes related to the embodiment will be described.

Implementation Configuration 1

Implementation Configuration 1 is an optical sensor probe measuring a flow velocity through a laser Doppler method, in which a pressing force of the optical probe is controlled such that it is 10 gw/mm² or less.

Examples of the method for controlling the pressing force of the optical sensor probe with high accuracy include a method for controlling a pressing force using stress against a strain of an elastic body such as a rubber body or a spring. For example, when the elastic modulus (Young's modulus) of an elastic body is E and the (compression) strain is T, the pressing load F is F=F-T. At this time, fluctuation of the pressing force during the measurement time becomes measurement noise, and therefore, it is desirable that the load be constant and static. In addition, it is desirable that the required pressing force for measuring the velocity of a fluid such as blood in capillaries of the soft scalp be 10 gw/mm² or less. In addition, it is desirable that the pressing force for a human body or the like excluding rigid parts such as the skeleton be as low as 5 gw/mm² or less.

Furthermore, the pressing force is controlled using the optical probe described in Implementation Configuration 1 to measure the flow velocity according to the principle of the laser Doppler method. In the case of measuring the velocity of a fluid such as blood in capillaries of the soft scalp, it is possible to perform measurement having a large signal-to-noise intensity ratio by measuring the flow velocity through a laser Doppler method while maintaining the contact between the measurement object and the distal end of the optical probe and maintaining the pressing force to such a degree that the movement of blood or the like in capillaries of the scalp is not hindered.

Implementation Configuration 2

Implementation Configuration 2 is an optical probe in which a change in pressing force due to a buckling length of optical fibers is used to adjust the pressing force of the optical fibers. That is, in Implementation Configuration 2, the pressing force of the optical fibers at a distal end of the optical fibers can be adjusted to 10 gw/mm² or less with high accuracy by adjusting the buckling length of the optical fibers.

In addition, Implementation Configuration 2 is a flow velocity measurement method in which it is possible to perform measurement having a large signal-to-noise ratio by using a pressing force due to fiber buckling and measuring the flow velocity through a laser Doppler method while maintaining the contact between a measurement object and a distal end of the optical probe and maintaining the pressing force so as not to hinder the movement of blood or the like in capillaries of the scalp.

In addition, in Implementation Configuration 2, at least one light-incident optical fiber may be used as the optical fiber. For light reception, scattered light from a measurement object may be received by installing a semiconductor photodetector near a measurement area.

Implementation Configuration 3

In addition, in Implementation Configuration 3, a plurality of optical fibers or a multi-core fiber are used as optical fibers used for an optical probe in a laser Doppler method.

In Implementation Configuration 3, it is possible to reduce production costs using one or more of the same optical fibers as a laser light-incident light guide and a light guide for guiding scattered light from a measurement fluid in the laser Doppler method. In addition, in a case where a multi-core fiber having a plurality of cores in an identical fiber is used in Implementation Configuration 3, the one optical fiber can be used for both laser light incidence and reception of scattered light from a measurement object.

Implementation Configuration 4

In addition, Implementation Configuration 4 is an optical probe of which a pressing force is controlled using a change in pressing force due to buckling of optical fibers, in which a plurality of optical fibers are used at a distal end of the optical probe and a core center interval is set to 500 μm or more and 1,500 μm or less.

Optical Penetration Depth and Core Center Interval Between Two Fibers

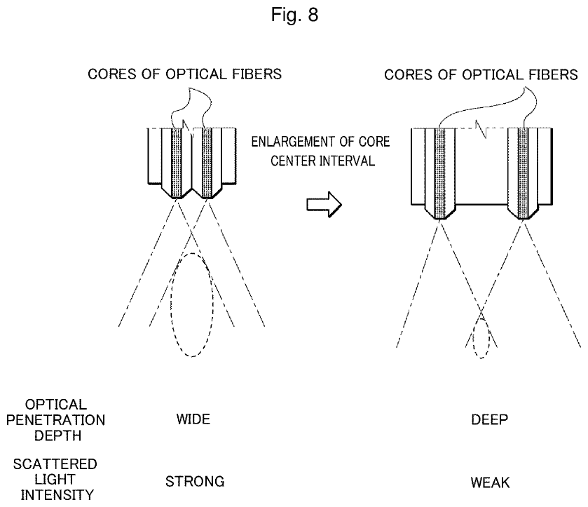

FIG. 8 shows a schematic diagram of a distribution of laser light incident on a measurement sample emitted from one optical fiber and a distribution of scattered return light which is scattered from the measurement sample and received at the other optical fiber in a case of using two sets of optical fibers having different core center intervals.

As shown in FIG. 8, it is necessary for laser light to spread from an emission end of the optical fiber for light transmission with a specific aperture angle and be incident on a measurement object and for scattered light from the measurement object to optically couple to the optical fiber for light reception having a specific aperture angle. Therefore, it is thought that an area where the distributions of the laser light and the scattered return light overlap is a main measurement area.

Accordingly, in a case where the interval between core centers of two fibers for light emission and light reception is narrow (left side in FIG. 8), many parts of the measurement areas overlap, and the light intensity of the scattered return light becomes stronger. However, the light penetration depth becomes wider from a shallow area to a deep area. At this time, the light intensity of the scattered return light becomes stronger in a case where the light penetration depth is shallow.

On the other hand, in a case where the interval between core centers of two fibers for light emission and light reception is wide (right side in FIG. 8), the scattered return light is only from an area with a deep light penetration depth, and the light intensity of the scattered return light also becomes weak.

That is, when measuring a blood flow rate, the signal intensity of the blood flow rate is maximized at a certain core center interval due to the relationship between the depth position of a blood vessel and the light intensity of scattered return light. In other words, the depth of an area where a blood flow rate is measured can be changed by changing a core center interval of two fibers for light emission and light reception.

Implementation Configuration 5

In addition, Implementation Configuration 5 is a flow velocity measurement method using an optical probe of a laser Doppler method, in which laser light having a plurality of light wavelengths is used as a light source with which a measurement object is irradiated. The plurality of light wavelengths can be used simultaneously or selectively.

Specifically, in a case where a blood flow in capillaries in subcutaneous tissue is considered as a measurement object, measurement of blood flow rates with laser light which has a wavelength of about 780 nm to 830 nm and a small difference in light absorption between red blood cells having adsorbed oxygen and deoxygenated red blood cells and with laser light having a large difference in light absorption can be performed simultaneously or alternately to separately evaluate the flow rate of the red blood cells having adsorbed oxygen.

In addition, in a case where it is desired to improve the signal-to-noise (intensity) ratio, by performing measurement of a flow rate through a laser Doppler method using different wavelengths of light A having a wavelength at which the light A is easily scattered by a structure (stationary tissue in the skin) of a measurement object and light B having a wavelength at which the light B is easily scattered by a fluid component in blood or the like, a flow rate Va using the light A mainly depends on shaking of the structure (stationary tissue in the skin) of the measurement object during measurement and a flow rate Vb using the light B becomes a vector sum of the fluid component in blood or the like and the structure (stationary component in the skin) of the measurement object. Therefore, the difference Vb-Va becomes a required flow rate of the fluid component in blood or the like in the measurement object.

For this reason, the measurement method is useful in a case where shaking of a structure (stationary tissue in the skin) of a measurement object affects measurement of a flow rate, for example, a case where shaking of a structure (stationary tissue in the skin) of a measurement object has the same speed order as that of a flow rate.

Embodiment of Optical Sensor Probe of Present Invention

Hereinafter, an embodiment of an optical sensor probe will be described in detail with reference to the drawings.

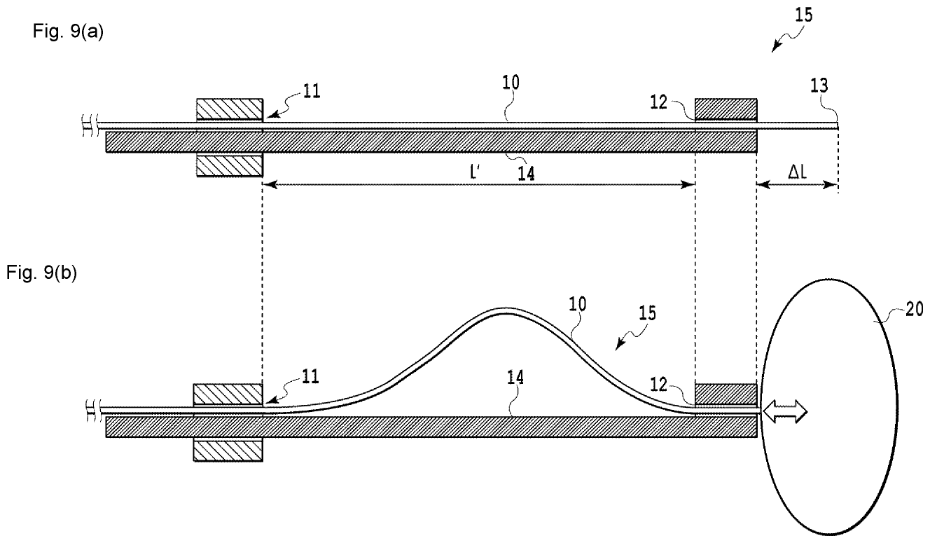

FIG. 9 is a schematic diagram showing a specific example of the embodiment of the optical sensor probe 15 of the present invention. Similar to FIGS. 5 and 6, FIG. 9(a) is a non-measurement state of the optical sensor probe 15 and FIG. 9(b) is a measurement state of the optical sensor probe 15.

The schematic diagram of FIG. 9(a) is the same as that of FIG. 6. However, the rod-shaped fiber support portion 14 is fixed by penetrating through two pipe-shaped tools separated by an adjustable distance L′, and the fibers 10 are linearly supported along the fiber support portion 14. A fixing portion 11 formed of the pipe-shaped tools on an optical probe base portion side can adjust the fixation position. The optical sensor probe 15 capable of adjusting a pressing force by adjusting a buckling length L′ by making the fixation position of optical fibers adjustable is produced.

In FIG. 9(b), the pipe-shaped tool on the pinhole (restriction hole) 12 side may be fixed to the fiber support portion 14, or may be formed as an integral structure with the fiber support portion 14. However, the optical fibers 10 are not fixed to the pinhole (restriction hole) 12. In order to prevent the scalp from being in a blood-blocking state (a state where a blood flow is blocked) during measurement, it is desirable that a cross-sectional portion of the pipe of the pipe-shaped tool (the member forming the restriction hole) on the pinhole (restriction hole) 12 which comes into contact with the scalp of the measurement target 20 have a shape, for example a flange shape, in which the contact area (cross-sectional area of the pipe) is enlarged as much as possible, and it is desirable that the surface of the flange coming into contact with the scalp be made of or covered with a flexible member.

The fixation position of the pipe-shaped tool of the fixing portion (fiber fixing point) 11 in FIG. 9 can be moved horizontally along the fiber support portion 14 for adjusting a pressing force. The pipe-shaped tool in the fixing portion 11 can be arbitrarily fixed to the fiber support portion 14 together with the optical fibers 10 at a position of a buckling length L′ generating a desired pressing force using, for example, a set screw or a locking mechanism. Since the pressing force required for the optical sensor probe is not large, an elastic member, for example, a rubber member, having a frictional force may be inserted into the pipe of the pipe-shaped tool of the fixing portion 11 so as to fix the pipe-shaped tool to the fiber support portion 14 together with the fibers 10 using the frictional force and to adjust the fixation position (buckling length L′) through a direct operation with the human hand.

By fixing one of the two pipe-shaped tools at an adjustable position and pressing the other one on the optical sensor probe distal side against a measurement sample in this manner, the optical fibers themselves buckle in the area of the buckling length L′ and an adjustable pressing force is generated. At this time, although it is necessary to fix the optical fibers on one side as a fixing point and to restrict movement of the optical fibers other than that of linear movement at a position at which the optical fibers pass through the hole on the other side, other conditions are not limited to having the form shown in the schematic diagram of FIG. 9, and there is no problem irrespective of the materials used for production thereof.

Furthermore, the pressing force of the optical sensor probe is generated by a bending moment of a cylindrical elastic body of optical fibers in principle. However, it is unnecessary to generate a pressing force through buckling of optical fibers, and a distal end of optical fibers can be pressed using a pressing force generated by a bending moment of a member of any material or shape as long as the same degree of pressing force can be generated.

In addition, compression or elongation stress of an elastic body such as a spring or rubber can be used without using the buckling of optical fibers or the like as long as the same degree of pressing force can be generated. A pressing force can also be generated using an energy conversion element that can convert energy such as heat or electricity of a piezoelectric element or the like into mechanical energy.

In addition, in the case of using the buckling force, quartz fibers having a large Young's modulus are desirable as optical fibers to obtain a sufficient pressing force, but the quartz fibers may be polymer-coated fibers, plastic optical fibers, or hole-assisted fibers. In addition, one or more fiber types may be used at one time.

In addition, it is desirable to use optical fibers having a graded index core in which hardly any propagation loss is caused due to buckling, but optical fibers having a step index core, multi-core fibers, PANDA optical fibers, or photonic crystal fibers may be used.

Furthermore, it is also possible to directly mount a laser diode (LD) for light emission or a photodiode (PD) for light reception at a distal end of an optical sensor probe as long as it is a mechanism for generating a pressing force through methods other than the buckling force of optical fibers as described above.

Form of Optical Sensor Probe with Single Optical Fiber

Laser light can be made to be incident on a measurement target such as the skin and scattered light therefrom can be received by optical fibers to measure a blood flow rate through a laser Doppler method using such an optical sensor probe.

At this time, it is desirable that an optical fiber that makes light be incident on a measurement object such as the skin and an optical fiber that receives scattered light be separate optical fibers in an optical sensor probe. However, it is also possible to realize an optical sensor probe in which the fiber buckling is utilized with only one optical fiber by making light be incident on a measurement object with one of the polarizations TE and TM and receiving scattered light from the measurement object with the other polarization using a PANDA optical fiber or the like.

Requirements for Light Source

In addition, the laser Doppler method uses an optical beat signal (optical Doppler effect) generated by interference between scattered light from stationary tissue in the skin and scattered light from red blood cells in blood as described above. Therefore, a light source such as a light emitting diode (LED) or amplified spontaneous emission light (ASE) can be used under the condition of a signal-to-noise (intensity) ratio in some measurements. However, since the light source is required to have coherence in addition to low frequency fluctuation and high light intensity stability, it is desirable to use a laser light source. In addition, in a case where a blood flow in blood vessels is considered, it is desirable that the wavelength be about 780 nm to 830 nm as described above.

In addition, a single-longitudinal-mode (SLM) laser or the like is more desirable from the viewpoint of capable of obtaining a single-longitudinal-mode beam without mode hopping or the like that causes a remarkable output change.

Hereinafter, the measurement method for a blood flow rate using the optical sensor probe of the embodiment of the present invention will be more specifically described, but the present invention is not limited to these examples.

Dependence of Blood Flow Rate on Pressure in Middle Finger Pad

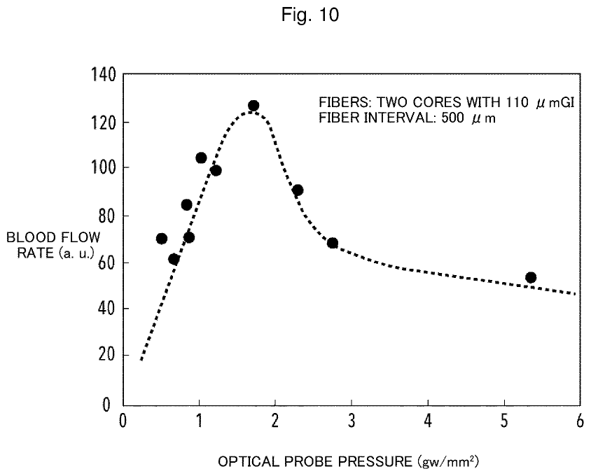

FIG. 10 shows measurement results of dependence of a blood flow rate on a probe pressure measured at a position of the middle finger pad of the left hand of a human body using the optical sensor probe of the embodiment of the present invention. This measurement is performed through a laser Doppler flow rate measurement method using the optical sensor probe shown in FIG. 9 in which two GI optical fibers having an outer diameter of 250 μm are respectively used as fibers for laser light incidence and scattered light reception by fixing distal ends of the GI optical fibers so as to have a fiber center interval of about 500 μm. The position of the middle finger pad of the hand is an area of the human body with a large blood flow rate and it is easy to measure the blood flow rate. Therefore, the blood flow rate is measured at the position first.

As a result, as shown in FIG. 10, it can be seen that the measurement values of the blood flow rates increase at first as the probe pressure increases, but the measurement values gradually decrease as the probe pressure increases after a maximum value of the blood flow rate at a probe pressure of around 1.7 gw/mm² in terms of pressure.

That is, it can be seen that there is an optimum value of a pressing force at which the measurement value of a blood flow rate is maximized in measurement of a blood flow rate of capillaries in the subcutis of a human body.

Dependence of Blood Flow Rate on Pressure in Left and Right Foreheads

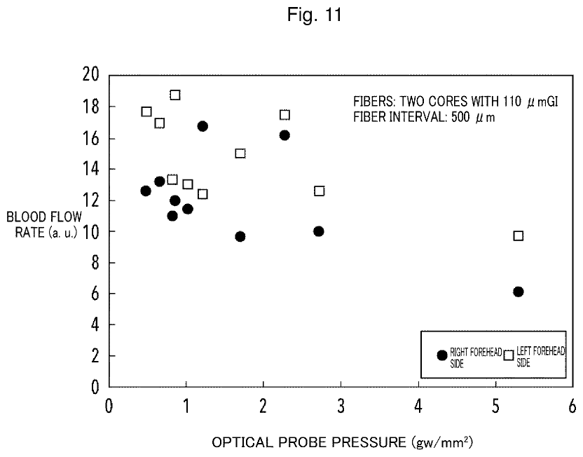

FIG. 11 shows measurement results obtained by measuring a blood flow rate in the left and right forehead of the human head similarly using the optical sensor probe of FIG. 9 under the same conditions as in FIG. 10.

In FIG. 11, • is a measurement value of dependence of a blood flow rate on a probe pressure at a position on the right forehead side of the human head, and □ is a measurement value of dependence of a blood flow rate on a probe pressure at a position on the left forehead side of the human head.

As a result, although there are variations, even the blood flow rates in the forehead part are maximized in the range of a weak probe pressure of 1 gw/mm² to a strong probe pressure of 2 gw/mm².

Dependence of Blood Flow Rate on Pressure at Top of Head

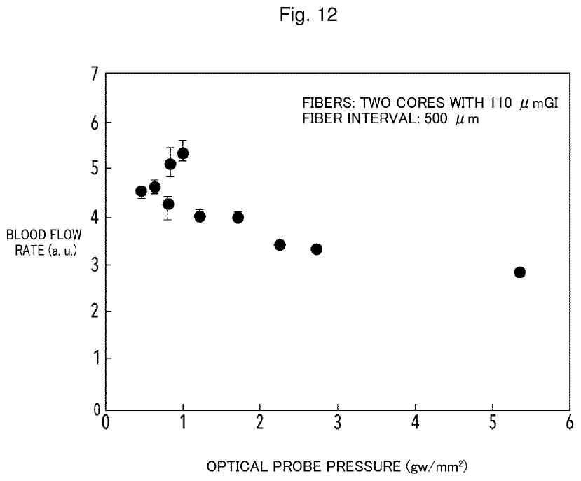

FIG. 12 shows measurement results obtained by measuring a blood flow rate at the top of the head under hair similarly using the optical sensor probe of FIG. 9 under the same conditions as in FIGS. 10 and 11.

In the measurement results of dependence of a blood flow rate on a probe pressure at a position of the top of the head under hair of the human head of FIG. 12, there are variations, but the blood flow rates at the top of the head are maximized in the range of a weak probe pressure of 1 gw/mm² to a strong probe pressure of 2 gw/mm².

Dependence of Blood Flow Rate on Optical Fiber Core Interval

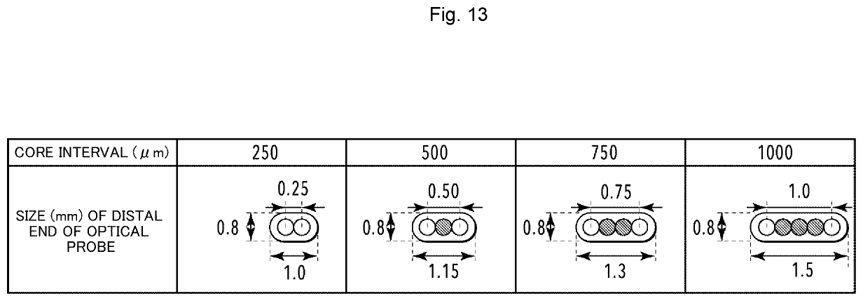

FIG. 13 shows cross-sectional views of four optical sensor probes produced for measuring the dependence of a blood flow rate on an optical fiber core interval, the cross-sectional views being perpendicular to a core optical axis in a distal portion of the optical fiber.

In the optical sensor probes shown in FIG. 13, 0 to 3 other optical fibers having the same diameter are arranged between an optical fiber for light incidence and an optical fiber for light reception arranged at both ends in a cross-sectional width direction, and are fixed with an adhesive. Accordingly, four sets of optical sensor probes having four different core center intervals of 250 to 1,000 μm between two optical fibers for light emission and light reception from distal ends of the probes were produced.

The four optical sensor probes in which a set of two optical fibers for light emission and light reception was used and which have different core center intervals had the structure of FIG. 9, and blood flow rates were respectively measured at the middle finger pad and the center of the forehead in a state where the pressing forces at the distal ends of the optical probes were constant at 1.7 gw/mm %.

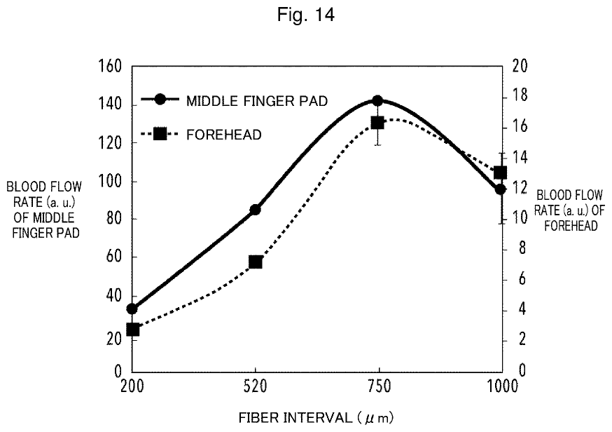

FIG. 14 shows results obtained by plotting the obtained measurement results of two sets of four points of each blood flow rate at the middle finger pad and the center of the forehead in order of fiber core center intervals. As a result, very similar changes were shown in both the middle finger pad and the center of the forehead, and the maximum value was 750 μm.

Switching Fiber or Core

In the cross-sectional view of FIG. 13, the optical fibers sandwiched between the two optical fibers for light emission and light reception at both ends do not transmit light and are represented by so-called dark fibers. However, the optical fibers sandwiched therebetween may transmit light. In this case, if a light source or a Doppler measurement device is provided with an light-switching function for switching fibers or cores, at least one of optical fibers for laser light emission and scattered light reception can be selectively switched to switch core intervals, and the depth of an area of the skin to be measured can be switched according to the principle illustrated in FIG. 8. In addition, if one fiber is used for laser light emission and other fibers are used as fibers for scattered light reception, an optical sensor probe having different optical fiber intervals as shown in FIG. 8 can be obtained simultaneously, and scattered light in an area having different light penetration depths can also be simultaneously measured.

For example, in FIG. 13, in the case of the probe which has a structure with a core interval of 1,000 μm and in which five optical fibers in total are used, it is possible to realize switching between four core intervals of 250 to 1,000 μm by selectively switching two out of five optical fibers and to measure blood flow rates in an area with different depths of the skin. Furthermore, it is also possible to simultaneously measure scattered light at the four core intervals of 250 to 1,000 μm. In addition, similarly in the case of a multi-core fiber, cores can be switched or scattered light can be simultaneously received.

Adapter Socket

In addition, two optical fibers for light emission and light reception at the distal portion of an optical sensor probe are exposed, and a member having a plurality of holes for inserting the exposed optical fibers can be attached to the probe as an adapter socket. The adapter socket of a probe may be formed as a member which has at least two holes with different intervals at both ends and in which a plural kinds of different cross sections perpendicular to cores of optical fibers as shown in FIG. 13 are formed. Alternatively, one adapter socket may be provided with a plurality of holes through which exposed optical fibers are to be inserted to selectively use the holes.

In this case, the cross section along the cores of the optical fibers of the adapter socket can be formed in a shape similar to the cross sections shown in FIG. 8. The interval between the two holes of the adapter socket may be widened or narrowed toward a distal end from the insertion part of the exposed optical fibers so that core center interval between the two optical fibers is widened or narrowed toward the end.

Such a plurality of adapter sockets having different widths can be prepared, distal ends of the two exposed optical fibers can be inserted therethrough, and the adapter sockets can be attached to an optical sensor probe. By doing so, the blood flow rates of the skin having different depths can be measured by changing the core center interval between the two optical fibers by exchanging only the adapter sockets of the probe without providing a light source or a Doppler measurement device with the light-switching function. In a case where a plurality of adapter sockets which have the same core center interval between two optical fibers and have different contact areas coming into contact with a measurement portion such as the skin are prepared, a pressing force on the measurement portion per unit area can be changed by changing the adapter sockets. Furthermore, in a case where an adapter socket to which an electrode for measuring pulse, a temperature sensor, or the like is attached in a part coming into contact with a measurement portion such as the skin is used, it is also possible to simultaneously measure pulse, body temperature, or the like simultaneously with the blood flow rate.

Measurement Method for Blood Viscosity or Vascular Elastic Modulus

Next, an embodiment of a measurement method for blood viscosity or a vascular elastic modulus using an optical sensor probe described above will be described.

Blood Flow State of Human Body

Regarding blood flow state at each area of a human body, the Reynolds number of a blood flow in the ascending aorta having a diameter of 20 to 32 mm is about 3,600 to 5,800, the Reynolds number of a blood flow in the descending aorta having a diameter of 16 to 20 mm is about 1,200 to 1,500, the Reynolds number of a blood flow in a thick artery having a diameter of 2 to 6 mm is about 110 to 850, the Reynolds number of a blood flow in the vena cava having a diameter of 20 mm is about 630 to 900, the Reynolds number of a blood flow in a thick vein having a diameter of 5 to 10 mm is about 210 to 570, and the Reynolds number of a blood flow in capillaries having a diameter of 0.005 to 0.01 mm is about 0.0007 to 0.003.

For this reason, the Reynolds number in the arterial and the veins substantially having a diameter of less 5 mm is sufficiently smaller than 2,000. Here, a fluid in a flow path (blood vessel) of which the Reynolds number is sufficiently smaller than 2,000 which is a critical Reynolds number has laminar flow. It is known that, in the case of the viscous fluid which has a Reynolds number sufficiently smaller than the critical Reynolds number (<2,000) and flows as laminar flow, the following equation is established.

That is, when the blood flow rate is Q, the blood viscosity is n, the radius of a blood vessel is r, the length of a blood vessel is 1, and the dynamic pressure (pressure difference between both ends of a blood vessel) is P, the following Hagen-Poiseuille relational equation is established.

$$Q = \frac{π {r}^{4}}{8 η l} \cdot P \qquad ( 6 )$$

Evaluation Method of Blood Viscosity

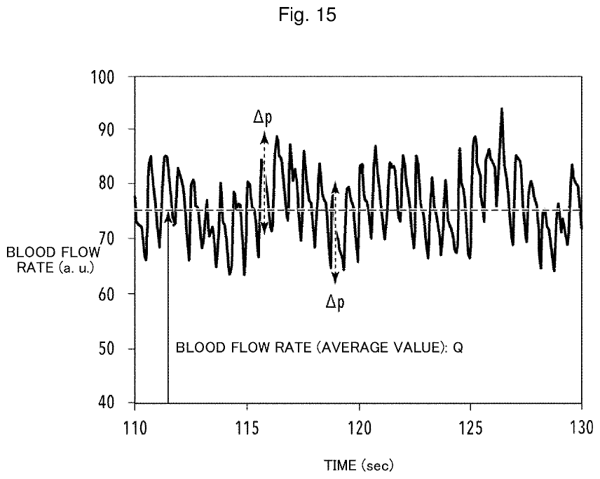

FIG. 15 shows a temporal change of a blood flow rate in the middle finger pad portion of a human body. The blood flow rate is not constant over time, and a pulse wave due to pulsation of the heart and blood flow rate fluctuation in a time cycle longer than that of the pulsation overlap. At this time, the blood flow rate difference Δp caused by the blood flow rate fluctuation in a short period of time in FIG. 15 may be considered as substantially constant because the pressure fluctuation due to pulsation of heart contraction is the main influence.

Here, in the Hagen-Poiseuille relational equation of Equation (6), the dynamic pressure P is caused by the pressure of the pulsation of the heart, and therefore, is proportional to the pulse wave amplitude Δp.

Accordingly, when the proportionality constant is C, P=C-AP is established. Therefore, the blood viscosity η is represented by Equation (7) below using Equation (6).

$$η = \frac{π C {r}^{4}}{8 l} \cdot \frac{Δ p}{Q} \qquad ( 7 )$$

That is, an indicator of the viscosity η of flowing blood can be obtained by a ratio Δp/Q of the pulse wave amplitude Δp to the blood flow rate Q.

From the above, regarding the embodiment of the measurement method for blood viscosity in the present invention, a pressing force of an optical sensor probe is first changed to actually measure the Docket No. 22120.89 blood flow rate Q and the pulse wave amplitude op several times. Based on the result, the ratio of the blood flow rate to the pulse wave amplitude can be obtained to obtain an indicator of the blood viscosity η from Equation (7).

Such an indicator of the blood viscosity also consequently includes influence such as other parameters such as a cross-sectional area in blood, and therefore, cannot be an absolute indicator of the blood viscosity alone. However, it is possible to calculate and relatively evaluate blood viscosity by measuring the blood flow rate and the pulse wave while changing the pressing force using an optical sensor probe, pressing subcutaneous blood vessels using the pressing force, and changing the cross-sectional area in the blood vessels.

The density of blood or the like in an identical subject is a parameter that is unlikely to change locally. Accordingly, it is possible to relatively measure the change in hardness of blood vessels by continuously measuring an identical area of an identical subject and continuously evaluating an indicator corresponding to a vascular elastic modulus.

Change in Blood Flow Rate due to Pressing

By pressing blood vessels, to which no pressing force has been applied, while gradually applying a pressing force thereto, cross-sectional area of the blood vessel tract and the flow rate decrease. When the pressing force is further increased, the cross-sectional area of the blood vessel tract eventually becomes 0 and blood does not completely flow, whereby a blood-blocked state is reached.

In addition, when the pressing force is decreased from the blood-blocked state, that is, a state where the blood flow rate is zero due to a strong pressure applied to the blood vessel to be measured, the blood flow increases according to the size of the cross-sectional area in the blood vessel. That is, it can be seen that the blood flow rate and the pressing force are correlate with each other in principle and depend on the size of the cross-sectional area in the blood vessel which changes due to elasticity of the blood vessel.

Evaluation Method for Vascular Elastic Modulus

Here, when the flow rate of blood is Q, the flow rate coefficient is a, the cross-sectional area in the blood vessel is A, and the flow velocity is V, the relationship between the flow velocity and the flow rate of blood flowing inside the blood vessel is represented by Equation (8) below.

Q=α×A×V (8)

At this time, it is thought that blood flows due to a pressure change Δp of a pulse wave according to Bernoulli's theorem. Therefore, when the density of blood is p, the flow velocity of blood is given by Equation 9 below.

$$V = \sqrt{\frac{2 Δ p}{ρ}} \qquad ( 9 )$$

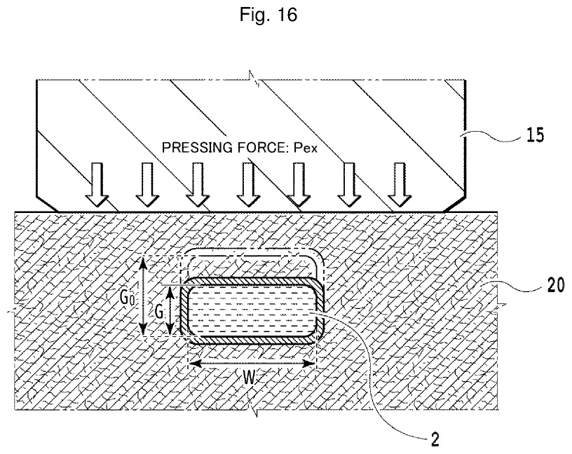

Here, as shown in FIG. 16, a state where a distal end of the fiber sensor probe 15 applies a pressing force Pₑₓ to the surface of the skin can be considered.

At this time, the blood vessel 2 is pressed by pressing of the probe. Since it is thought that the distal end of the fiber sensor probe 15 has a sufficiently larger cross-sectional area than the subcutaneous capillary 2, the pressing force Pₑₓ perpendicularly applied to the cross section of the subcutaneous blood vessel is uniform. At this time, it is thought that the distal end of the optical sensor probe is sufficiently harder than that of the blood vessel 2 and the subcutaneous blood vessel is deformed in a form along the planar shape of the distal end of the optical sensor probe 15. When it is assumed that the cross section is deformed like the blood vessel of FIG. 16, the cross-sectional area A in the blood vessel can be approximated to a rectangular shape with a gap G and a width W, and the blood flow rate Q is represented by the following equation.

$$Q = α GW \sqrt{\frac{2 Δ p}{ρ}} \qquad ( 10 )$$

At this time, the blood vessel 2 is pressed by the external pressing force Pₑₓ in FIG. 16. It is thought that, as primary approximation, the change in width W of the blood vessel is almost negligibly small and only the thickness G (the height of the gap between upper and lower inner walls in the drawing) of the blood vessel changes.

On the other hand, in a case where the thickness of the blood vessel when the external pressing force Pₑₓ is 0 is G₀ and the vascular elastic modulus (Young's modulus) is E, the external pressing force Pₑₓ is represented by the following equation according to Hooke's law.

Pₑₓ=E(G₀−G) (11)

That is, since G=G₀−Pₑₓ/E is established, Equation 12 below is obtained from Equation 10.

$$\frac{Q}{{ }^{\sqrt{Δ p}}} = α W \sqrt{\frac{2}{ρ}} ( {G}_{0} - \frac{{P}_{ex}}{E} ) = ( α W {G}_{0} \sqrt{\frac{2}{ρ}} ) - ( \frac{α W}{E} \sqrt{\frac{2}{ρ}} ) \cdot {P}_{ex} \qquad ( 12 )$$

That is, it can be seen that, when the above-described primary approximation is carried out, the ratio (the left side of Equation 12) of the blood flow rate Q to a square root of the pulse wave Δp is determined as a linear function of the external pressing force Pₑₓ and the inclination is inversely proportional to a vascular elastic modulus E.

Accordingly, regarding the embodiment of the measurement method for a vascular elastic modulus in the present invention, the pressing force of the optical sensor probe is first changed to actually measure the blood flow rate and the pulse wave plural times. Based on the measurement result, the relationship between the ratio of the blood flow rate to the square root of the pulse wave and the change in pressing force is obtained. By quantifying dependence (inclination) of the ratio of the blood flow rate to the square root of the pulse wave on the pressing force, it is possible to obtain an indicator corresponding to a reciprocal of the vascular elastic modulus E.

Here, the pressing force Pₑₓ is not applied only to the blood vessel 2 as shown in FIG. 16. The elastic modulus E of Equation 11 is also affected by elastic moduli of other than blood vessels or subcutaneous tissue. In a case of considering a correlation with the blood flow rate and the pulse wave amplitude in Equations 10 and 12, there is no problem in that the elastic modulus E is an elastic modulus of a blood vessel (and around the blood vessel that affects blood flow) related to the ratio of the blood flow rate to the square root of the pulse wave amplitude (the elastic modulus which is uncorrelated with the blood flow rate and the pulse wave amplitude is a constant item of Equation 12.)

In addition, the indicator corresponding to the reciprocal of the vascular elastic modulus E also includes influence such as other parameters such as the density of blood, and therefore, cannot be an absolute indicator of the vascular elastic modulus alone. However, the density of blood or the like in an identical subject is a parameter that is unlikely to change locally. Accordingly, it is possible to relatively measure the change in hardness of blood vessels by continuously measuring an identical area of an identical subject and continuously evaluating an indicator corresponding to a vascular elastic modulus.

Examples of Measurement Method

Examples of the measurement methods for blood viscosity and a vascular elastic modulus of the present invention will be more specifically described, but the present invention is not limited to these examples.

### Example 1 of Measurement Method: Evaluation of Blood Viscosity of Middle Finger Pad

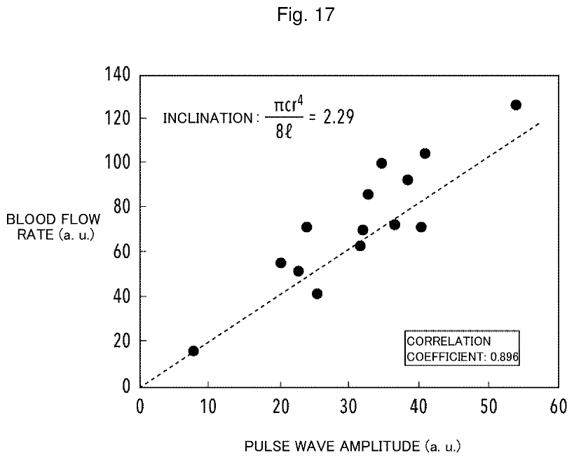

FIG. 17 is a diagram showing dependence of a blood flow rate on a pulse wave amplitude measured at a position of the middle finger pad of the left hand of a human body using the optical sensor probe of the embodiment of the present invention for evaluating blood viscosity.

In this measurement, the optical sensor probe which was shown in FIG. 9 and in which only distal ends of two GI optical fibers each having an outer diameter of 125 μm were fixed while having a fiber center interval of about 500 μm was used. Two fibers were used as fibers for light incidence and light reception to measure a blood flow rate through a laser Doppler flow rate measurement method. The position of the middle finger pad of the hand is an area of the human body with a large blood flow rate and is an area where it is easy to measure the blood flow rate.

As a result, the measurement intensity of the blood flow rate increased as the pulse wave amplitude increased as shown in FIG. 17. It was found that the pulse wave amplitude and the measurement intensity of the blood flow rate tended to be in a proportional relationship. Therefore, as a result of performing an approximation calculation through linear approximation, the correlation coefficient was 0.896 showing a strong positive correlation, and a correlation suitable for the Hagen-Poiseuille relational equation as shown in Equation 7 was obtained.

In addition, the inclination of a straight line of the linear approximation corresponding to a reciprocal of blood viscosity was calculated using Equation 7, and the result showed a value of 2.29. A relative indicator corresponding to the blood viscosity η was obtained using this value.

### Example 2 of Measurement Method: Evaluation of Blood Viscosity of Left and Right Forehead of Head

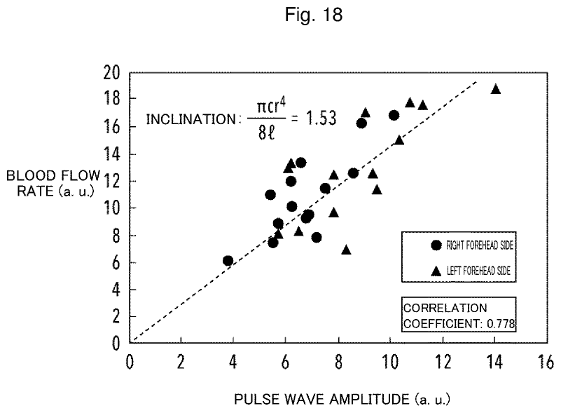

FIG. 18 is a diagram showing dependence of a blood flow rate on a pulse wave amplitude measured at positions on the left and right forehead of the head using the optical sensor probe of the embodiment of the present invention for evaluating blood viscosity.

This measurement was also performed under the same conditions as those in Example 1 using the optical sensor probe shown in FIG. 9.

As a result, it was found that the pulse wave amplitude and the measurement intensity of the blood flow rate tended to be in a proportional relationship even in Example 2 of the measurement method as shown in FIG. 18, and a positive correlation with a correlation coefficient of 0.778 was recognized. Since the positions on the left and right forehead of the head are measurement areas having a smaller blood flow rate than the middle finger pad of Example 1, a decrease in correlation coefficient due to measurement errors was observed. The inclination of the straight line of the linear approximation obtained was 1.53.

Such inclinations of straight lines of linear approximation are relative indicators corresponding to blood viscosity n, and there is a possibility that the thickness of a subcutaneous blood vessel at a measurement area may vary. Therefore, the identical measurement area can be periodically measured to grasp a relative change in blood viscosity.

### Example 3 of Measurement Method: Evaluation of Blood Viscosity Using Ratio of Blood Flow Rate to Pulse Wave at Top of Head

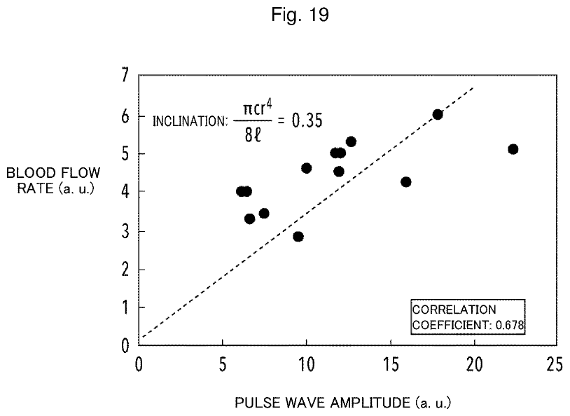

FIG. 19 is a diagram showing dependence of a blood flow rate on a pulse wave amplitude measured at the top of the head in the embodiment of the optical sensor probe of the present invention for evaluating blood viscosity.

This measurement was also performed under the same conditions as those in Example 1 of the measurement method using the optical sensor probe shown in FIG. 9. FIG. 19 shows the dependence of a blood flow rate on a pulse wave amplitude at the position of the top of the head under hair of the human head.

As a result, it was found that the pulse wave amplitude and the measurement intensity of the blood flow rate tended to be in a proportional relationship even in Example 3 of the measurement method as shown in FIG. 19, and a positive correlation with a correlation coefficient of 0.678 was recognized. Since the position of the top of the head is a measurement area where the blood flow rate is smaller than those of Examples 1 and 2, a decrease in correlation coefficient due to measurement errors was observed. The inclination of the straight line of the linear approximation obtained was 0.35.

Such inclinations of straight lines of linear approximation are relative indicators corresponding to blood viscosity η, and there is a possibility that the thickness of a subcutaneous blood vessel at a measurement area may vary. Therefore, the identical measurement area can be periodically measured to grasp a relative change in blood viscosity.

### Example 4 of Measurement Method: Evaluation of Vascular Elastic Modulus at Middle Finger Pad and Top of Head

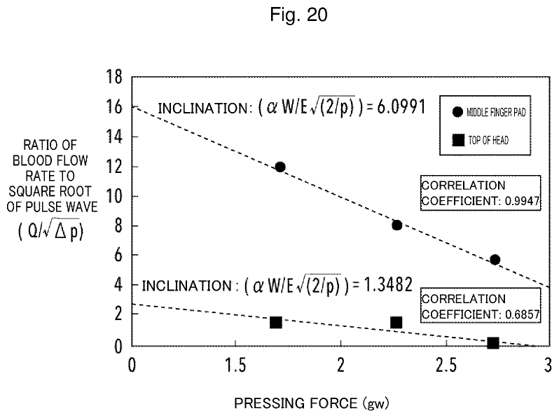

FIG. 20 shows dependence which is a result obtained by measuring a ratio of a blood flow rate to a square root of a pulse wave amplitude by changing a pressing force of a distal end of an optical sensor probe for evaluating a vascular elastic modulus.

This measurement was also performed under the same conditions as those in Example 1 of the measurement method using the optical sensor probe shown in FIG. 9.

In this measurement, the pressing force (the horizontal axis of FIG. 20) was changed in three ways at two positions of the middle finger pad of the hand and the top of the head to perform the measurement. The ratio Q/√(Δp) (the vertical axis of FIG. 20) of a blood flow rate to a square root of a pulse wave amplitude was calculated based on the obtained measurement values.

As a result, linear changes as shown in Equation 12 were shown at both the middle finger pad and the center of the forehead as shown in FIG. 20. In particular, the value of the inclination shown as a coefficient

$$( \frac{α W}{E} \sqrt{\frac{2}{ρ}} )$$

of the pressing force Pₑₓ of Equation 12 at the middle finger pad was about 6.1.

The correlation coefficient at this time was 0.9947 showing an extremely favorable linear correlation. On the other hand, the value of the inclination shown at the top of the head was about 1.35. The correlation coefficient at this time is 0.6857, which does not show such a high linear correlation. It is thought that this is because of influence of measurement errors due to a small number of measurements of a blood flow rate or a pulse wave amplitude.

These inclination values are indicators that directly depend on the vascular elastic modulus E. However, since the state of a blood vessel at a measurement area varies, the vascular elastic modulus at the identical measurement area can be periodically measured to grasp a change in vascular elastic modulus over time.

### INDUSTRIAL APPLICABILITY

As described above, according to the measurement method of the present invention in which the optical sensor probe of the present invention is used, it is also possible to evaluate a vascular elastic modulus and blood viscosity using measurement results of a pulse waveform and a blood flow rate obtained by adjusting pressing with high accuracy. For this reason, the present invention can be used in various industrial fields such as preventive medicine or initial treatment and has an extremely large industrial utility value.

### REFERENCE SIGNS LIST

1 Scalp
2 Blood vessel (capillaries)
3 Skull
4 Red blood cell
5 Laser light
6, 7 Scattered light
10 Optical fiber
11 Fiber fixing point (fixing portion)
12 Pinhole (restriction hole)
13 Distal end of optical fiber
14 Fiber support portion
15 Pressing optical probe (optical sensor probe)
20 Measurement target (skin)

1. An optical sensor probe comprising: optical fibers of which one end is connected to a light source or a Doppler measurement device measuring a flow velocity using a laser Doppler, wherein, at the other end, the optical fibers are linearly supported and arranged in a section of a buckling length L in a case where an optical sensor probe is in a non-measurement state, fixed to a fiber fixing point on an optical fiber-proximal side in the section of the buckling length L so that a movement of the optical fibers is restricted thereat, and arranged so that a distal end of the optical fibers protrudes through a restriction hole on an optical fiber-distal side in the section of the buckling length L, and wherein, at the other end, in a case where the optical sensor probe is in a measurement state, the distal end of the protruding optical fibers abuts on a measurement target, the optical fibers are allowed to move only in a linear direction connecting the fiber fixing point to a center of the restriction hole by a protrusion length ΔL when pushed through the restriction hole, and the optical fibers in the section of the buckling length L buckle and maintain a pressing force between the measurement target and the distal end of the optical fibers by an elastic force.

2. The optical sensor probe according to claim 1, wherein, when a fiber diameter of the optical fibers is d, the protrusion length ΔL is smaller than a threshold value ΔLc given by an equation below, and the pressing force between the distal end of the optical fibers of the optical sensor probe and the measurement target can be adjusted with high accuracy by adjusting the protrusion length ΔL of the optical fibers.

3. The optical sensor probe according to claim 1, wherein, when the fiber diameter of the optical fibers is d, the protrusion length ΔL is larger than a threshold value ΔLc given by an equation below, and the pressing force between the distal end of the optical fibers of the optical sensor probe and the measurement target can be adjusted with high accuracy by adjusting the buckling length L of the optical fibers.

4. The optical sensor probe according to claim 1, wherein a plurality of optical fibers or multi-core fibers are used as the optical fibers.

5. The optical sensor probe according to claim 4, wherein the light source or the Doppler measurement device is provided with a light switching function or a multiple simultaneous measurement function so that at least one fiber or core for light emission or light reception can be switched, or two or more optical fibers can perform simultaneous light emission or light reception.

6. The optical sensor probe according to claim 4, wherein at least one optical fiber at the distal portion of the optical sensor probe is exposed, and a member having at least one hole for inserting the exposed optical fibers can be attached to the optical sensor probe as an adapter socket.

7. The optical sensor probe according to claim 1, wherein a plurality of light wavelengths are simultaneously or selectively used.

8. A measurement method for a blood flow rate, the method comprising: measuring a Doppler shift of scattered light from the measurement target due to light emitted from the light source, using the Doppler measurement device to measure the blood flow rate of the measurement target using the optical sensor probe according to claim 1.

9. A measurement method for blood viscosity, the method comprising: measuring the blood viscosity from the blood flow rate and a pulse wave amplitude which change due to the adjustment of the pressing force of the optical sensor probe in the measurement method for a blood flow rate according to claim 8.

10. The measurement method for blood viscosity according to claim 9, the method comprising: controlling a buckling length of the optical fibers and changing the pressing force of the optical sensor probe at the distal end of the optical fibers to measure the blood viscosity from a ratio of the blood flow rate to the pulse wave amplitude which changes.

11. A measurement method for a vascular elastic modulus, the method comprising: adjusting and changing the pressing force of the optical sensor probe to measure a vascular elastic modulus from the obtained blood flow rate and pulse wave amplitude in the measurement method for a blood flow rate according to claim 8.

12. The measurement method for a vascular elastic modulus according to claim 11, wherein the vascular elastic modulus is measured from a proportional coefficient of a relationship between a ratio of the blood flow rate to a square root of the pulse wave amplitude and the pressing force of the optical sensor probe.
